(19) United States
(12) Patent Application Publication
(10) Pub. No.: US 2022/0153506 A1
(43) Pub. Date: May 19, 2022

(54) Method and Apparatus for Storage and Delivery of Test Strips

(71) Applicants: Alan J. Leszinske, Albuquerque, NM (US); Thomas L. Tucker, Star Valley Ranch, WY (US)

(72) Inventors: Alan J. Leszinske, Albuquerque, NM (US); Thomas L. Tucker, Star Valley Ranch, WY (US)

(21) Appl. No.: 17/526,913

(22) Filed: Nov. 15, 2021

Related U.S. Application Data
(60) Provisional application No. 63/113,859, filed on Nov. 14, 2020.

(51) Int. Cl. B65D 83/08; G01N 33/487

(52) U.S. Cl. CPC B65D 83/0829; B65D 2583/082; G01N 33/48757

(57) ABSTRACT

An easy to use, low-cost dispenser container delivers individual test strips ready for insertion into a glucometer without requiring extraction of individual strips manually from the package. The system may provide a dry, contaminant-free, storage device whereby stored strips are not repeatedly exposed to humidity. As each strip is removed from the device, a gasket seals the storage, Desiccants may be used to maintain the integrity of the storage environment. A convenient built-in waste receptacle for used strips may include a mechanical means for removing the used strips from the glucometer without the need for touching the bloodied end of the used strips.

### CLAIM OF PRIORITY

The present application includes subject matter disclosed in and claims priority to a provisional application entitled “Method and Apparatus for Storage and Delivery of Test Strips” filed Nov. 14, 2020 and assigned Ser. No. 63/113,859, describing an invention made by the present inventors, herein incorporated by reference.

### BACKGROUND OF THE INVENTION

### 1. Field of the Invention

The present invention relates to handling, storage, and/or disposal, of diagnostic measurement test strips, such as the glucose test strips utilized by consumer diabetics and healthcare professionals in clinical settings. The present invention can be utilized for a variety of test strips measuring different analytes, individually or multiple analytes simultaneously, whose presence is monitored h medical health professionals and/or non-professionals who, due to medical conditions, must test liar the presence or absence thereof.

### 2. Description of Related Prior Art

Two factors applicable to the measurement of non-compliance for diabetics are inconvenience and cost. The background of the invention is to provide an innovative solution that alleviates some of the inconvenience without adding significantly to the cost.

A primary objective is to facilitate the presentation/delivery of individual test strips for insertion into the respective electronic measurement device (glucometer) from a dry and contaminant free storage environment without exposing all of the remaining unused test strips in the container to the external environment or the potential fur spillage and/or contamination of those remaining strips, as well as the removal and disposal of said strips. It is understood that opening a vial of test strips and repeatedly exposing it to moisture and other contaminants in the environment can negatively impact the accuracy (and therefore efficacy) and shelf-life of chemically based test strips.

One of the main goals, in the case of diabetics, is to eliminate the need for manually attempting to extract individual strips from current packaging vials given the progressive complications of diabetes winch include impaired vision, loss of sensitivity in hands and fingers, and diminished hand-eye coordination.

Exacerbating this problem is the drive to make strips smaller and smaller, it the difficulty for diabetics, and thereby, decreasing adherence to necessary measurement/monitoring compliance required for diabetics to appropriately control their blood sugars, which has been demonstrated to delay or slow the progression of the insidious and devastating complications associated with diabetes. In addition, there is no convenient means or place for disposal of used test strips.

In hospital/clinical settings similar issues arise for different reasons. Healthcare professionals and care providers wearing, gloves experience similar difficulties extracting and handling individual strips for insertion into clinically utilized glucometers (and other strip-based diagnostic equipment—cholesterol, glycated hemoglobin, cardiac markers, etc.).

Another primary advantage is the convenience of a built-in\self-contained containers for waste disposal, alleviating the need for finding a trash receptacle or picking the strip up off the floor after missing the receptacle. Having a ready and easily accessible means for disposal of the used strips will alleviate some of this inconvenience. A mechanical means for engaging the protruding. end of the used test strip and mechanically removing it from the glucometer and depositing it into the built-in waste receptacle without the need for physical contact with the bloodied end of the used test strip.

A number of devices and methods have been described in the prior art for packaging, storing, and dispensing diagnostic test strips. In most cases the devices described are more complex and are expected to be more costly than the invention described herein, and none appear to be readily available for purchase in the consumer or clinical markets.

### SUMMARY OF THE INVENTION

An easy to use, low-cost (injection molded plastic and/or 3D printing materials), disposable device/apparatus that delivers individual strips ready for insertion into the glucometer (or other measurement devices) by consumer diabetics or healthcare professionals without having to extract individual strips manually from the package. In addition, the apparatus provides a dry, contaminant-free, storage device whereby stored strips are not repeatedly exposed to humidity and other environmental contaminants or the potential for spilling the entire vial. Strips may be stacked longitudinally in the housing. As each strip is removed from the device a gasket seals the storage, and the top of the apparatus is sealed with a fitted cap and includes, in various forms, humidity-mitigating materials to maintain the integrity of the storage environment. A convenient built-in waste receptacle for used strips and a mechanical means for removing the used strips from the glucometer without the need for touching the bloodied end of the used strips.

Briefly described, and in an embodiment chosen for illustration, a stack of test strips rests upon a platform forced upward by a spring pressuring the stack against the top to hold the element with a slot in one end sufficiently large for finger/thumb contact to be made with the top-most strip in the stack. The strip is frictionally moved forward by the thumb/finger to extrude the electrode portion of measurement device. Upon insertion of the electrodes in the glucometer, the rest of the strip is drawn from the device and the gasket seal the slit. A fitted cap is placed over the top of the device to seal out moisture and any other potential contaminants. After the test strip in the glucometer has been utilized to measure the blood, the used strip is mechanically removed from the testing device and deposited in the built-in waste container on the side or bottom of the device. The remaining store of unused strips is securely contained within the storage area of a disposable plastic dispenser in an environment free of contamination from external sources (moisture, humidity, . . . ). Humidity-mitigating materials (as provided by some desiccants) are included, such as below, loose or in pouches added to the apparatus, coated upon, or incorporated into the housing of the apparatus.

In one preferred embodiment, the unit is wholly disposable, would be provided sealed, with the strips preloaded, and would not be refillable. Alternative embodiments allow for the removal and replacement of the slotted top piece and some means of refilling the strip magazine with the slotted top being replaced while external pressure was applied to the freshly loaded stack of test strips. In such an embodiment, a means, would also be provided for emptying, and resealing the waste strip receptacle or replacing it altogether with a new waste strip disposal unit.

In an alternative embodiment, the slotted top is replaced with a top piece that includes a slider that contacts the edge or posterior end of the topmost strip and propels it forward without external contact or exposure. In this embodiment the slide would be operated by finger or thumb to advance the electroded end of the strip through the gasket-sealed slot for insertion into the measurement device (glucometer). The slide would have a contact surface or protrusion for engagement by the thumb/finger and a means of frictionally or mechanically advancing the top-most test strip when engaged, without displacing the strips below or exposing them to the external environment or potential contaminants therefrom. Housing may include longitudinal grooves or extending shelves in the side walls to provide a track for guiding the mechanism.

In an alternative embodiment, the slotted top is replaced with a top piece that includes a slider that contacts the upper surface of the top-most strip via a high friction compound (elastomeric or other compositions) imposed upon the lower surface of the slider and propels it forward without external contact or exposure. In this embodiment the “slide” is operated by a finger or thumb to advance the electroded end of the strip through the gasket sealed slot for insertion into the measurement device (glucometer).

The upper side of the slide would include a physical protuberance or a high-friction contact surface that may also be coated, or partially coated, with a coating, comprising elastomeric or Other high-friction mechanically advancing the topmost test strip when engaged, without displacing the strips below or exposing them to the external environment or potential contaminants therefrom.

In a preferred embodiment, the waste receptacle includes a grasping mechanism for engaging the bloodied end of the used test strip allowing the used strip to push into the receptacle tot storage and later disposal from its posterior (electroded) end, without touching the bloodied end of the strip.

In a preferred embodiment, the waste receptacle includes a grasping mechanism and slider for engaging the bloodied end of the used test strip and mechanically drawing it completely into the receptacle for storage and later disposal without touching the used test strip.

In a preferred embodiment, the strip dispenser and waste receptacle are designed as cartridges for insertion into a resealable easement comprising metal, plastic, and/or other composite that provides a robust and resealable barrier to moisture, humidity-mitigation, airborne contaminants and other interfering agents or compounds. Such housing for the dispenser and waste cartridges can be designed in a variety of geometric alignments including side-by-side, stacked, back-to-back, inverted side-by-side in accordance with end-user preferences related to ergonomics and/or aesthetics.

### BRIEF DESCRIPTION OF THE DRAWINGS

The present invention will be described with greater specificity and clarity with reference to the fc Mowing drawings, in which:

FIG. 1 illustrates an exploded side view of a strip dispenser of an embodiment of the present invention.

FIG. 2 illustrates a frontal perspective view of a dispenser in a case of an embodiment of the present invention with cap open.

FIG. 3 illustrates a rear cross-sectional view of a strip dispenser and waste receptacle of an embodiment the present invention.

FIG. 4 illustrates a front view of a stacked dispenser and waste receptacle of an embodiment of the present invention.

FIG. 5 illustrates a side cross-sectional view of a stacked dispenser and waste receptacle of an embodiment of the preset invention.

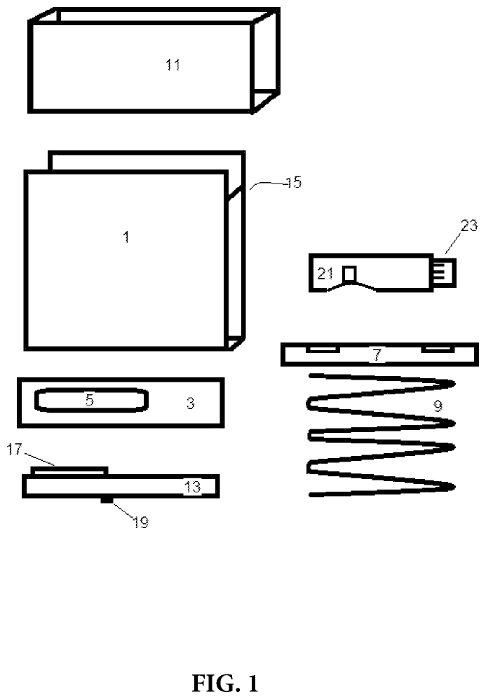

### DETAILED DESCRIPTION OF THE INVENTION

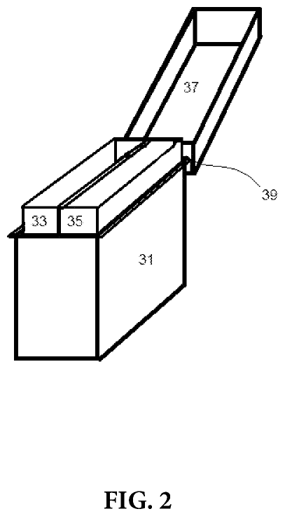

In one preferred embodiment of the device, the apparatus is comprised of a container/device housing (1), platform (7), and slotted top piece (3) through which the user s thumb, finger, high friction contacting slider, or slider with an engaging apparatus/protrusion can make contact with the topmost test strip (21) from the stack and advance the electroded end of the test strip (23) through the dispensing slot (5) for insertion into the electronic measurement device for the analyte of interest (e.g., a glucometer). The dispenser housing, platform and top piece (slotted or other embodiment) can be manufactured of plastic, metal, composite materials, or 3-D printing composites that ma include moisture/humidity mitigating, properties. A spring (9) that may be metal or other material that provides sufficient upward force to push the stack of test strips upwards against the slotted top piece (3) resides under the platform (7), and whose opposite end is butted against the inside bottom surface of the device housing (1). Humidity and other potential contaminants are excluded from the storage area by the gasket sealed slot (15) in the device housing (1), moisture/humidity mitigating materials/compositions, and augmented by is the form fitted, gasketed, deformable cap (11), and/or a resealable casement in FIG. 2 (33) that covers and seals the entire top of the device including the dispensing slot (5).

In another preferred embodiment, a slide (13) resides under the slotted top piece (3) and sits on top of the stacked test strips. The slide (13) will have an upwardly protruding thumb/finger engagement protrusion or high-friction surface/surface coating (17) that extends through the dispensing slot (5) and a downwardly protruding strip engagement protrusion (19) that engages the trailing edge of the top-most test strip in the stack and when moved forward advances the electroded end of the strip through the dispensing slot (5) for insertion into the glucose meter or other electronic measurement device.

In one preferred embodiment, the dispenser platform (1) and waste receptacle (43) are housed in a side-by-side orientation within a resealable casement (31) in FIG. 2 with an environmental moisture barrier fitted cover (37) (snap cap) attached to said casement by an external hinge (39). The casement (31) contains individual and separate housing areas for the dispenser (33) and the waste receptacle (35) that is enclosed by the common environmental moisture barrier fitted cover (37).

In another preferred embodiment, the dispenser platform (1) and waste receptacle (43) are attached via impermanent adhesive or other material (Velcro®) in a side-by-side orientation within a resealable easement (31) in FIG. 2 with an environmental moisture barrier fitted cover (37) (snap cap) attached to the casement for the dispenser platform (I) by an external hinge (39). The casement (31) contains a housing area solely for the dispenser (33) providing an enclosure for the dispenser with an environmental moisture barrier fitted cover (37). In this embodiment, the waste receptacle can be easily removed and replaced, and a replacement or refillable dispenser unit (33) can be inserted/reinserted into the casement (31).

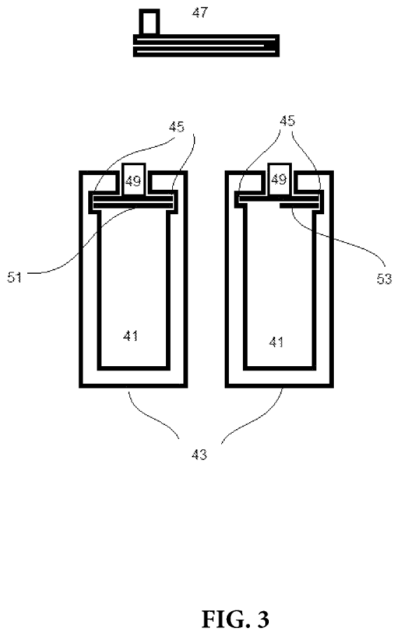

In one preferred embodiment in FIG. 3, the waste receptacle (43) comprises a storage area (41) with fitted closure and a grasping mechanism (47) incorporated in the waste receptacle (43). The grasping mechanism is for engaging the bloodied end of the used test strip and allowing the user to advance the used strip into the waste receptacle storage area (43) manually from the posterior (electrode) end of the used strip. The grasping mechanism (47) includes a pincer top (49) and pincer bottom (51) incorporated in the anterior wall of the waste receptacle. (43). When the pincer top (49) is depressed the used test strip is engaged between the pincer top (49) and pincer bottom (51), holding the used test strip in place while it is pulled from the monitoring device and user manually advances the used strip into the waste container (43) by pressing on the now exposed posterior (electrode) end of the used test strip.

In one preferred embodiment in FIG. 3, the waste receptacle (43) includes a storage area (41) with fitted closure and slider grooves (45) for a fitted grasping slider cover (47). Incorporated in the grasping slider cover (47) includes a mechanism for engaging the bloodied end of the used test strip and drawing it into the waste receptacle storage area (43). The grasping slider cover (47) includes pincer top (49) and pincer bottom (51) incorporated in the grasping sliding, cover (47). When the pincer top (49) is depressed, the used test strip is engaged between the pincer top (49) and pincer bottom (51), both of which are then drawn by thumb or finger towards the snap cap environmental barrier (37) until the used strip is drawn completely into the waste container (43). Pressure is then removed from the pincer top (51) releasing the stop into the waste receptacle storage area (41). The waste receptacle storage area (41) is closed by repositioning the grasping slider cover (47) away from the snap cap environmental barrier (37) until it is in the closed position. Pincer bottom designs (51) or (53) may be incorporated in preferred embodiments to facilitate release of the used test strip into the waste receptacle storage area (41).

In another preferred embodiment, the waste receptacle (43) may be manufactured as a stand-alone apparatus, or incorporated into a holding case/casement housing multiple waste receptacle units.

In another preferred embodiment, a plurality of the dispenser platform (1), distinctly colored or marked to indicate analyte to be measured, are housed in the resealable casement (31) in a plurality of geometric orientations, with an environmental moisture barrier fined cover (37) (snap cap(s)) attached to said casement by an eternal hinge (39) or hinges depending on geometric orientation. The casement (31) contains individual and separate housing areas for each of the dispenser platforms (33) that are enclosed by the common environmental moisture barrier fitted cover (37). The casement (31) and/or the individual dispenser platforms (33) will include desiccant pack, be coated/painted with moisture/humidity-mitigating materials such as provided by a desiccant, or be comprised of materials with moisture/humidity-mitigating materials incorporated or impregnated therein.

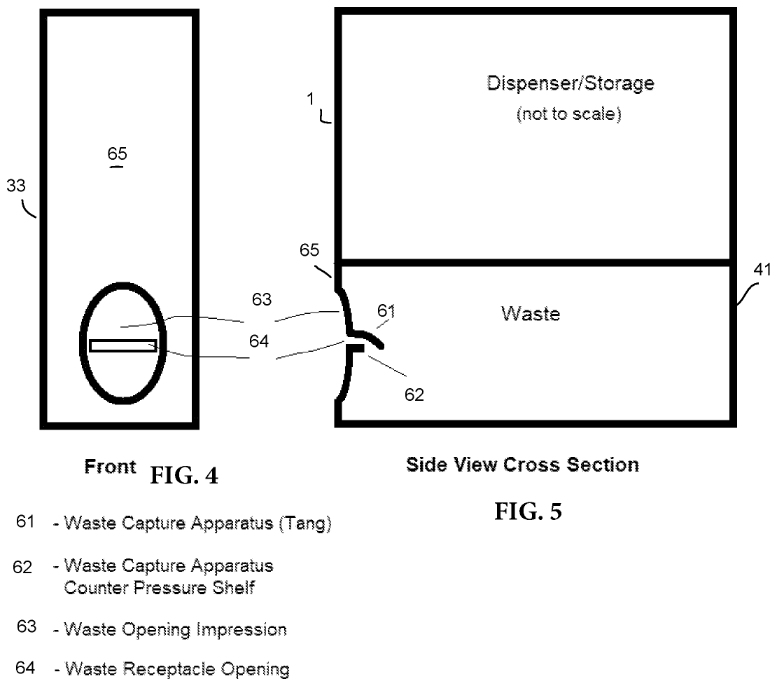

In a preferred embodiment, dispenser (1) and waste receptacle (41) may be stacked, joined, or formed of a single unitary body, as shown in FIGS. 4 and 5. Otherwise, the dispenser(s) and waste receptacle may be contained within a casement, or the like. Front of unitary both or casement (33) may include a was e receptacle opening (64). Waste receptacle opening may be set within a depressed opening impression (63) on a front wall (65) of the casement. Impression is preferably round, such as circular (partial spherical) in form, or oval-like as shown). Behind the opening, a waste capture apparatus, such as tang (61) is preferably a descending curved tab that at least partially or totally obscures the opening when viewed from the front. Tang may define the upper edge of the opening. Tang may be biased downwardly by a spring (not shown) or of a spring-type material (e.g., plastic, spring steel, etc.), that can be pressed upward by entering waste tabs. A counter-pressure shelf (62) may be set on the interior of the front wall, ear at the lower edge of the opening. Tang preferably extends rearward beyond shelf. Tang may occlude the opening, and may mate with a rear edge of the shelf, or otherwise hang thereover.

We claim:

1. An apparatus for housing and dispensing a plurality of stacked flattened objects, said apparatus comprising:
a rigid outer housing;
a platform upon which the plurality of stacked flattened objects is stacked, said plurality of stacked flattened objects stacked longitudinal a biasing mechanism directing said platform and a stack of flattened objects in an upward direction against the top of said housing;
said housing comprising:
a base; a top; and an integral dispensing side, disposing side, anterior, posterior, and interior walls forming a first containment area defining an interior volume for storing flattened objects longitudinally stacked on said platform; and a second containment area for discarding said flattened objects after their use;
said anterior and posterior walls, and said interior walls being spaced apart a distance greater than the respective length and width of the flattened objects.

2. The apparatus of claim 1 whereby the top of said housing comprises a slot or opening sized to allow a finger or thumb to pass through and optionally contact and advance for dispensing an individual flattened objects of said plurality of stacked flattened objects.

3. The apparatus of claim 1 whereby the top of said housing comprises a sliding mechanism adapted to engage and dispense an individual flattened object of said plurality of stacked flattened objects when said sliding mechanism is repositioned from an anterior position to a posterior position and then returned to its anterior position; said sliding mechanism engaging the test strips via an engagement mechanism/protuberance or a high-friction contact surface, or a high friction coating applied to a strip-contacting surface.

4. The apparatus of claim 3 whereby the interior walls of the apparatus contain longitudinal grooves forming a track for containing and guiding the sliding mechanism.

5. The apparatus of claim 1 whereby said apparatus is designed as a single-fill disposable.

6. The apparatus of claim 1 whereby said apparatus is designed to be refillable and reusable.

7. The apparatus of claim 1 whereby the housing is constructed of composition incorporating moisture/humidity-mitigating materials.

8. The apparatus of claim 1 whereby an internal surface of the housing or an internal surface of a casement set around said housing is coated, painted or impregnated with a desiccant.

9. The apparatus of claim 1 whereby said apparatus is manufactured, marked, or printed to distinctly and individually differentiate said housing with various distinct and separate analytes (e.g., color coding for glucose, protein C, troponin, etc.).

10. The apparatus of claim 1 further comprising a waste receptacle attached to a bottom of said housing, said waste receptacle comprising: a plurality of flat walls, said plurality of flat walls comprising side walls and forming a contained area for the storage of waste; said waste receptacle optionally attached to said housing via a plurality of optional geometric alignments, permanently or via impermanent adhesive.

11. The apparatus of claim 10 whereby said waste receptacle is not attached to the housing, and said waste receptacle being housed adjacent said housing in a holding case or casement.

12. The apparatus of claim 10 whereby said waste receptacle being unattached to the housing; said waste receptacle positioned in a holding case or casement.

13. The apparatus of claim 10 wherein said waste receptacle is adapted to receive used test strips manually discarded through a slot in an end-wall of the waste receptacle.

14. The apparatus of claim 10, wherein said waste receptacle comprises a mechanical means for engaging a bloody end of a used test strip and removing the used test strip from the monitoring testing device, and said means allowing the used test strip to be manually advanced into the waste receptacle through pressure applied to its posterior (electrode) end.

15. The apparatus of claim 10, wherein said waste receptacle comprises a mechanical means for engaging a used test strip and drawing the used test strip into the contained by means of sliding mechanism that is guided by longitudinal grooves in the sidewalls of the waste receptacle.

16. A waste receptacle of claim 10, whereby the mechanical mechanism for engaging used test strips has upper and lower engaging arms of the same breadth.

17. The apparatus of claim 10, said waste receptacle comprising a mechanical means for engaging used test strips, said means comprising an upper and lower engaging arm of differing breadths to facilitate the release of used test strips into the waste containment area.

18. The apparatus of claim 10 further comprising a casement or holding case formed of a plurality of sides, said casement or holding case containing the housing and the waste receptacle in one of a plurality of geometric alignments/orientations.

19. The apparatus of claim 18, wherein said casement or holding case includes an external hinge mechanism that attaches and aligns a fitted cover for the casement.

20. The apparatus of claim 18, whereby said casement or holding case includes a fitted cover for a snap cap; said snap cap forming a moisture barrier to protect any interior contents from environmental elements.

21. The apparatus of claim 18 wherein said casement or holding case containing at least two of said housings.

22. The apparatus of claim 20 wherein at least one of said waste receptacle, casement or housing case, snap cap, or said housing comprising at least one of a plurality of materials selected from the group compromising plastics, composite materials, metals, and/or 3-D printing materials that may include incorporate moisture/humidity-mitigating properties.
